(19) United States
(12) Patent Application Publication
(10) Pub. No.: US 2024/0407724 A1
(43) Pub. Date: Dec. 12, 2024

(54) MEDICAL DIAGNOSTIC GARMENT

(71) Applicant: Xavier Atencio, Haines City, FL (US)

(72) Inventor: Xavier Atencio, Haines City, FL (US)

(21) Appl. No.: 18/503,806

(22) Filed: Nov. 7, 2023

Related U.S. Application Data
(60) Provisional application No. 63/471,849, filed on Jun. 8, 2023.

(51) Int. Cl. A61B 5/00; A61B 5/0205; A61B 5/029; A61B 5/1455; A61B 5/256; A61B 7/02; A61B 8/08

(52) U.S. Cl. CPC A61B 5/6804; A61B 5/02055; A61B 5/029; A61B 5/14552; A61B 5/256; A61B 7/026; A61B 8/0883

(57) ABSTRACT

A medical diagnostic garment includes a main body shirt having a plurality of diagnostic sensors that are positioned along the inside facing surfaces of the shirt for collecting patient data of a patient wearing the shirt. The sensors including a plurality of electrocardiogram sensors, pulse oxygen sensors, temperature sensors, ultrasound sensors and listening sensors. A controller is connected to each of the sensors for providing power and operating instructions. Patient data captured by the sensors is communicated to the controller. The controller includes functionality for communicating with an external device such as a smartphone running a mobile application and for conducting a video telemedicine session with a healthcare provider interface device.

### CROSS-REFERENCE TO RELATED APPLICATIONS

This application claims the benefit of U.S. Application Ser. No. 63/471,849 filed on Jun. 8, 2023, the contents of which are incorporated herein by reference.

### TECHNICAL FIELD

The present invention relates generally to medical diagnostic equipment, and more particularly to a diagnostic garment for use in medical examinations.

### BACKGROUND

The statements in this section merely provide background information related to the present disclosure and may not constitute prior art.

Traditionally, medical diagnostic instruments have been designed to perform a single function and to be used independently of other instruments. For this reason, medical practitioners such as nurses and doctors often employ multiple instruments when evaluating a patient for a particular illness or injury.

Recently, there has been an increase in the use of virtual medical appointments (referred to generally as telemedicine) which allows remotely located patients to interact with a healthcare provider for non-emergency care. Through the use of video, the provider is able to see and speak with the patient in order to see any obvious injuries but is unable to perform a thorough physical examination. As such, it is not uncommon for healthcare providers to ultimately refer telemedicine patients to a nearby medical facility to review vital statistics such as heart rate, blood oxygen levels, and/or to undergo a test such as an EKG, for example.

Although there are several types of non-prescription instruments on the market such as blood pressure kits, pulse oximeters, and stethoscopes, for example, the cost of obtaining each of these single-use instruments is high. Additionally, many users would be unable to properly use such instruments to convey results to a doctor. For example, if a patient is complaining of stomach pain, the patient would have a difficult time positioning the stethoscope along the intestines to listen for problems. Moreover, even if they were able to hear something, there is no way for the patient to convey the sounds to a provider during a telemedicine session.

Accordingly, it would be beneficial to provide a plurality of diagnostic instruments that are incorporated into a garment that can be worn by a patient so as to ensure each instrument is properly aligned with the patient's body to detect and record patient information. It would also be beneficial if the garment is capable of sending the patient information to a healthcare provider during a telemedicine session.

### SUMMARY OF THE INVENTION

The present invention is directed to a medical diagnostic garment. One embodiment of the present invention can include a main body in the form of a shirt having a front panel, a back panel, a neck area, a head opening, a waist opening and a pair of arm openings. A plurality of medical diagnostic sensors can be positioned along the inside facing surfaces of the shirt and can function to collect patient data of a patient wearing the shirt. A controller is connected to each of the sensors for providing power and operating instructions. Data captured by the sensors is communicated to the controller for storage or transmission to an externally located device.

In one embodiment, the plurality of sensors can include a plurality of electrocardiogram electrodes that are positioned along the garment, and the patient information includes information about the heart of the patient. In one embodiment, the plurality of sensors can include a pulse oxygen sensor that is positioned along the garment, and the patient data includes information about the pulse oxygen of patient.

In one embodiment, the plurality of sensors can include at least one temperature sensor that is positioned along the garment, and the patient data includes information about the body temperature of the patient. In one embodiment, the plurality of sensors can include at least one ultrasound sensor that is positioned along the garment, and the patient data includes a visual representation of an internal organ of the patient.

In one embodiment, the plurality of sensors can include at least one listening sensor that is positioned along the garment, and the patient data includes an audible sound produced by an internal organ of the patient.

In one embodiment, the controller includes functionality for connecting to a patient interface device to allow the patient or other user to control the operation of the sensors directly. In one embodiment, the device can include a mobile application for installation on the patient interface device to allow a voice or video telemedicine conference with a remotely located provider operating a provider interface device. During a telemedicine conference, the provider interface device can control the operation of the sensors.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a front view of the medical diagnostic garment, in accordance with one embodiment of the invention.

FIG. 2 is a back view of the medical diagnostic garment, in accordance with one embodiment of the invention.

FIG. 3 is a simplified block diagram of the system controller of the medical diagnostic garment, in accordance with one embodiment of the invention.

FIG. 4 is an exemplary operating environment of the medical diagnostic garment, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” means a series of identified physical components which are linked together and/or function together to perform a specified function.

As described throughout this document, the term “about” “approximately” “substantially” and “generally” shall be used interchangeably to describe a feature, shape or measurement of a component within a tolerance such as, for example, manufacturing tolerances, measurement tolerances or the like.

As described herein, the term “removably secured,” and derivatives thereof shall be used to describe a situation wherein two or more objects are joined together in a non-permanent manner so as to allow the same objects to be repeatedly joined and separated.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

As described herein, the term “patient data” and “patient information” are used interchangeably to include any type of data which can be captured, recorded, collected, discerned, inferred, or otherwise gleaned from one or more of the sensors of the garment. Several nonlimiting examples of patient data can include, but are not limited to: body temperature, pulse rate, respiration rate, blood oxygen saturation rate; electro cardiogram information, lung, intestine and heart sounds, among others, for example.

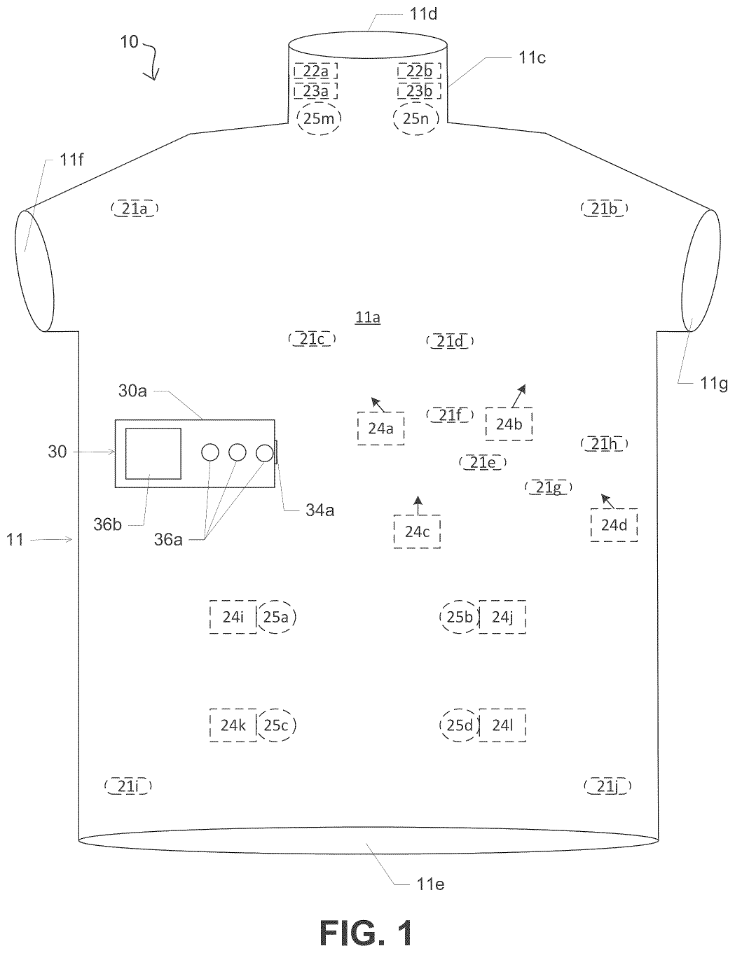

FIGS. 1-4 illustrate one embodiment of a medical diagnostic garment device 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

As shown in the drawings, the diagnostic garment device 10 can include, essentially, a garment 11, a plurality medical diagnostic sensors 21a-251, and a controller 30.

In one embodiment, the garment 11 can include or comprise a shirt having a shape that is complementary to the shape of a human user so as to position the below described sensors adjacent to the major organs, arteries and intestinal tracts of a wearer. In one embodiment, the shirt can include a front panel 11a, a back panel 11b, a neck area 11c, a head opening 11d, a waist opening 11e and a pair of arm openings 11g and 11f.

The shirt can be constructed from any number of different materials suitable for direct contact with human skin such as cotton, polyester and/or polycotton, among others, for example. The shirt can be manufactured in a wide variety of different sizes such as small, medium, large, and extra-large, among other sizes, for example, and can include any type of markings, decorative elements, and the like. For example, the garment can include alignment markings on the garment that are to be positioned over the wearers' nipples and navel to ensure the wearer has properly aligned the garment to their torso.

Because the fit and alignment of the garment is important to ensure the below described sensors are positioned at specific locations along the wearer's anatomy, it is contemplated that potential users may be pre-fitted for a specifically sized garment. To this end, the user may initially meet with a medical assistant or other individual who can take and record user measurements such as neck size, shoulder width, mid portion collar bone location, left and right breast bones, chest/bust circumference and/or waist circumference, for example. These measurements may also include cross-measurements between two or more of the identified body portions, among others, for example. Such a feature ensures the garment issued to the user will fit tightly (e.g., secure against the user's skin without wrinkles and shifting), thus positioning the sensors at the locations outlined below.

Although illustrated with regard to a shirt with short sleeves, this is for illustrative purposes only, as the shirt may also include long sleeves to permit one or more of the below described sensors to be positioned anywhere along the arms and wrists of a user. Additionally, the garment may also include, comprise or consist of lower body attire such as underwear, short pants or long pants, for example, each having additional sensors positioned therein to capture additional patient data. Finally, it is noted that the garment may comprise an entire body suit such as a short or long-sleeved jumpsuit, for example.

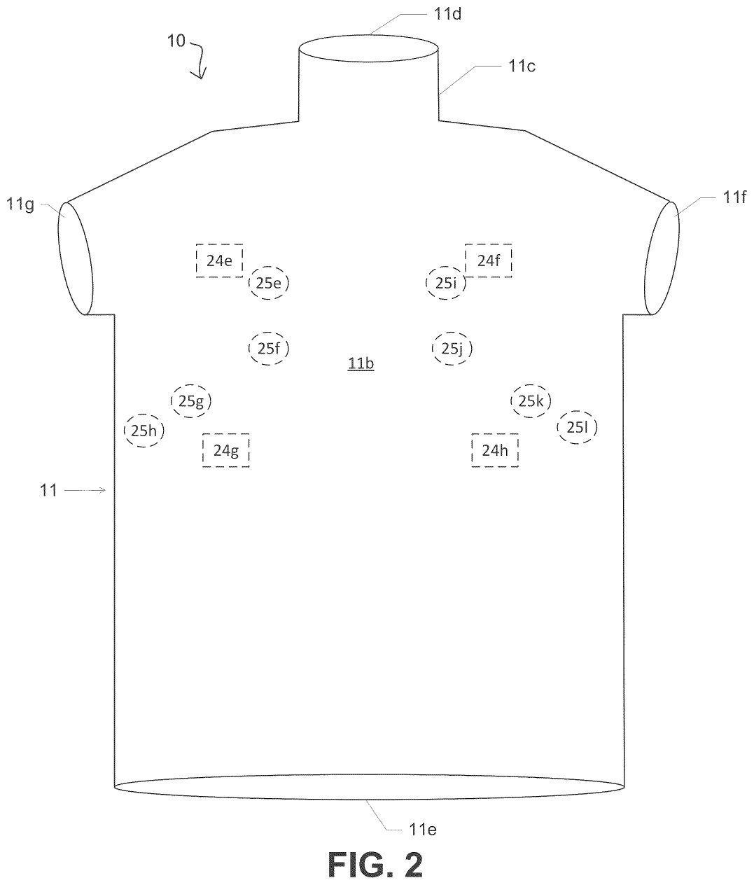

As shown best at FIGS. 1 and 2, one embodiment of the device 10 can include a plurality of medical diagnostic sensors for collecting patient data that are provided along and/or within the garment body 11. In the preferred embodiment, each of the below described sensors can be positioned along the inside facing surfaces of the garment so as to be in direct contact with the skin of the patient wearing the garment.

In one embodiment, the device 10 can include ten Electrocardiogram (ECG) electrode sensors 21a, 21b, 21c, 21d, 21e, 21f, 21g, 21h, 21i, and 21j, (referred to collectively as 21), that function to record the electrical signals that make the heartbeat. In the preferred embodiment, the electrodes can be positioned along the front surface 11a of the garment in accordance with standardized twelve lead ECG placements as provided by the published medical standards of the American Heart Association, and more particularly as outlined in the Recommendations For The Standardization And Interpretation Of The Electrocardiogram Location Of Standard Limb And Precordial Electrodes. As shown, the electrodes can be placed:
a. electrode 21a is positioned along the right shoulder of the wearer;
b. electrode 21b is positioned along the left shoulder of the wearer;
c. electrode 21c is positioned along the fourth intercostal space on the right sternum of the wearer;
d. electrode 21d is positioned along the fourth intercostal space at the left sternum of the wearer;
e. electrode 21e is positioned along the fifth intercostal space at the midclavicular line of the wearer;
f. electrode 21f is positioned midway between placement of electrodes 21d and 21e;
g. electrode 21g is positioned along the anterior axillary line on the same horizontal level as electrode 21f;
h. electrode 21h is positioned along the mid-axillary line on the same horizontal level as electrodes 21f and 21g;
i. electrode 21i is positioned along the lower right-side abdomen of the wearer; and
j. electrode 21j is positioned along the lower left side abdomen on the same horizontal level as electrode 21i.

Such positioning is critical so as to capture the electrical activity of the heart from multiple angles, to aid the practitioner in determining if the patient has arrhythmia, an acute infarct, and other issues. Of course, other embodiments are contemplated wherein a different number of ECG electrodes are provided at the same or other locations along the garment.

In one embodiment, the device 10 can include two pulse oxygen sensors 22a and 22b (referred to collectively as 22) that function to detect the saturation of oxygen carried in the red blood cells. In the preferred embodiment, the pulse oxygen sensors will be positioned bilaterally along the front surface 11a of the garment at the neck area 11c. Such positioning is important as this ensures the sensors are located adjacent to the left and right carotid arteries which provide blood to the brain. Of course, other embodiments are contemplated wherein a different number of pulse oxygen sensors are provided at the same or other locations along the garment.

In one embodiment, the device 10 can also include two temperature sensors 23a and 23b (referred to collectively as 23) that may also be positioned bilaterally along the neck area 11c. Each of the temperature sensors can function to detect the body temperature of a user wearing the garment. The use of two sensors is designed specifically to provide redundancy and accuracy. Of course, any number of other temperature sensors may be provided at different locations along the garment.

In one embodiment, the device 10 can include a first plurality of ultrasound sensors 24a, 24b, 24c, and 24d that are positioned along the front panel of the garment 11a, and a second plurality of ultrasound sensors 24e, 24f, 24g and 24h that are positioned along the back panel 11b. Each of the sensors 24a-24h can function to emit and capture sound waves that are designed to allow the physician to perform an echocardiogram of a user wearing the garment and to capture patient data such as how bold flows through the heart and heart valves of the patient, among other information.

In the preferred embodiment the first plurality of ultrasound sensors can be positioned along the front panel 11a as follows:
a. ultrasound sensor 24a is positioned along the sternum and oriented away from the heart to capture parasternal long axis views of the heart and arteries of the wearer;
b. ultrasound sensor 24b is positioned along the same horizontal level as sensor 24a, and is oriented toward the heart to capture parasternal short axis views of the heart and arteries of the wearer;
c. ultrasound sensor 24c is positioned below sensor 24a, and is oriented toward sensor 24a to capture subxiphoid views of the heart and arteries of the wearer; and
d. ultrasound sensor 24d is positioned beneath sensors 24a and 24b, is diagonal to sensor 24c, and is oriented toward sensor 24a to capture Apical views of the heart and arteries of the wearer.

In the preferred embodiment the second plurality of ultrasound sensors can be positioned along the back panel 11b as follows:
a. ultrasound sensor 24e is positioned at the left Mid-axillary line around the 6th and 7th intercostal space of the wearer;
b. ultrasound sensor 24f is positioned at the right Mid-axillary line around the 6th and 7th intercostal space of the wearer;
c. ultrasound sensor 24g is positioned on the left side: at the intersection of the posterior axillary line and rib space between the 10th and 12th rib of the wearer; and
d. ultrasound sensor 24h is positioned on the right side: at the intersection of the posterior axillary line and rib space between the 10th and 12th rib of the wearer.

Such positioning of the ultrasound sensors is critical so as to capture moving pictures and data pertaining to the functioning of the heart, heart valves and related arteries, to aid the practitioner in determining if the patient has heart damage, heart failure, clogged arteries, pleural fluid, pneumothorax, volume overload and the like, among other issues.

In one embodiment, the device 10 can include a third plurality of ultrasound sensors 24i, 24j, 24k, and 24l that are positioned along the lower portion of the front panel 11a of the garment to capture patient data pertaining to the intraabdominal area of the patient. Imaging two perpendicular lines that intersect at the umbilicus, dividing the abdomen in four different quadrants. We will refer to each quadrant based on upper, lower, left and right (based on the wearers own left and right orientation while facing forward). In the preferred embodiment the third plurality of sensors can be positioned along the back panel 11b as follows:
e. Ultrasound sensor 24i is positioned on the right upper quadrant, exactly in the center of that quadrant;
f. Ultrasound sensor 24j is positioned on the left upper quadrant, exactly in the center of that quadrant;
g. Ultrasound sensor 24k is positioned on the right lower quadrant, exactly in the center of that quadrant; and
h. Ultrasound sensor 24l is positioned on the left lower quadrant, exactly in the center of that quadrant.

Such positioning is critical to ensure the sensors 24i-241 are capable of capturing patient data such as moving pictures and data pertaining to the intraabdominal area such as the stomach, intestines, gallbladder and the like. Of course, other embodiments are contemplated wherein a different number of ultrasound sensors are provided at the same or other locations along the garment.

In one embodiment, the device 10 can include a first plurality of listening sensors such as microphones 25a, 25b, 25c, 25d and 25e, which can be positioned along the front panel 11a of the garment to capture bowel sounds from the user. Imaging two perpendicular lines that intersect at the umbilicus, dividing the abdomen in four different quadrants. We will refer to each quadrant based on upper, lower, left and right (based on the patient's own left and right orientation while facing forward). In the preferred embodiment the first plurality of listening sensors can be positioned as follows:
a. Listening sensor 25a is positioned on the right upper quadrant, exactly in the center of that quadrant, adjacent to 24i;
b. Listening sensor 25b is positioned on the left upper quadrant, exactly in the center of that quadrant, adjacent to 24j;
c. Listening sensor 25c is positioned on the right lower quadrant, exactly in the center of that quadrant, adjacent to 24k; and
d. Listening sensor 25d is positioned on the left lower quadrant, exactly in the center of that quadrant, adjacent to 24l.

Such positioning ensures the listening sensors are able to capture patient information pertaining to the sounds made by the intraabdominal area such as the stomach, intestines, gallbladder and the like. Of course, other embodiments are contemplated wherein a different number of listening sensors are provided at the same or other locations along the garment.

In one embodiment, the device 10 can include a second plurality of listening sensors 25e, 25f, 25g, 25h, 25i, 25j, 25k, and 251 that are positioned along the back panel 11b of the garment to capture patient data pertaining to the lungs of the patient. In the preferred embodiment the second plurality of sensors can be positioned along the back panel 11b as follows:
a. Listening sensor 25e is positioned Left Side: in the inner border of the scapula (shoulder blade) at the top;
b. Listening sensor 25f is positioned Left Side: in the inner border of the scapula (shoulder blade) in the mid portion of the bone;
c. Listening sensor 25g is positioned Left Side: in the inner border of the scapula (shoulder blade) in the lower portion of the bone;
d. Listening sensor 25h is positioned Left Side: right below the mid portion of the scapula (shoulder blade);
e. Listening sensor 25i is positioned Right Side: in the inner border of the scapula (shoulder blade) at the top;
f. Listening sensor 25j is positioned Right Side: in the inner border of the scapula (shoulder blade) in the mid portion of the bone;
g. Listening sensor 25k is positioned Right Side: in the inner border of the scapula (shoulder blade) in the lower portion of the bone; and
h. Listening sensor 25l is positioned Right Side: right below the mid portion of the scapula (shoulder blade).

Such positioning ensures the listening sensors are able to capture patient information pertaining to the sounds made by the lungs of the patient, such as wheezing, crackles, rhonchi, and the like, among other information. Of course, other embodiments are contemplated wherein a different number of listening sensors are provided at the same or other locations along the garment.

In one embodiment, the device 10 can include a third plurality of listening sensors 25m and 25n that are positioned along the front panel 11a at the neck 11c of the garment to be positioned adjacent to the carotid arteries. Such placement allows the sensors to capture patient data pertaining to the sound of blood flowing through the arteries.

Although described above with regard to specific types of sensors, numbers of sensors and placement of sensors along the inside surfaces of the garment, this is for illustrative purposes only. To this end, other embodiments are contemplated wherein the device includes any number of other types of sensors and/or number of sensors, which may be positioned at any number of different positions and surfaces of the garment.

Although not illustrated, other embodiments of the garment are contemplated that further include a Sphygmomanometer (blood pressure cuff) which can be positioned along the right sleeve of the garment. The device including an integrated inflation pump, power source and transducer which can be connected to the below described system controller. Such a feature permitting the garment to capture patient information that includes the blood pressure of the garment wearer.

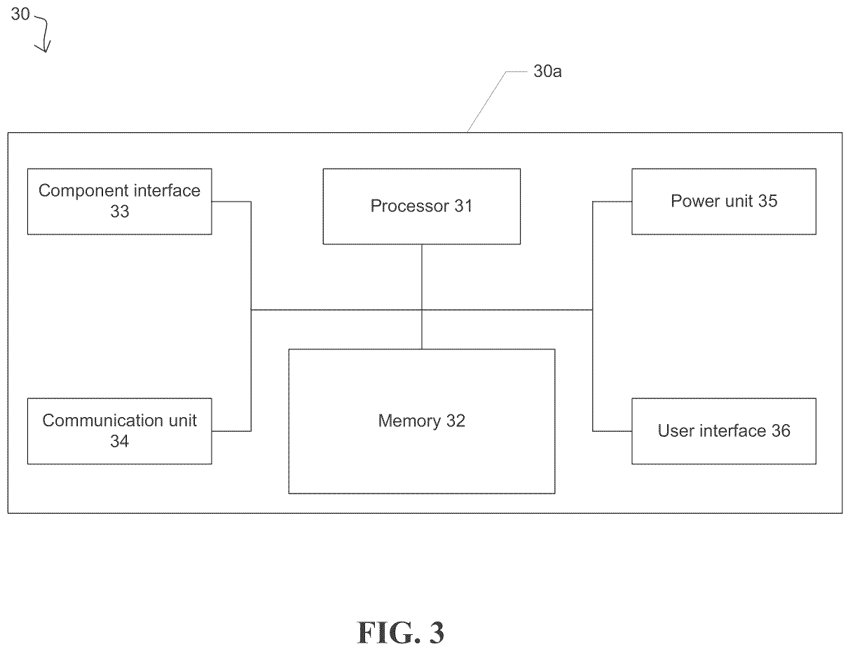

FIG. 3 is a simplistic block diagram illustrating one embodiment of the system controller 30. The controller can be in electrical communication with each of the plurality of sensors via internal communication cables, so as to allow the controller to operate each of the sensors to capture patient information and/or send the same to an external device.

In one embodiment, the controller 30 can include a processing unit 31 that is conventionally connected to an internal memory 32, a component interface unit 33, a communication unit 34, a power unit 35, and/or a user interface 36.

Although illustrated as separate elements, those of skill in the art will recognize that one or more system components 31-36 may include, comprise, or consist of one or more printed circuit boards (PCB) containing any number of integrated circuit or circuits for completing the activities described herein. The controller will preferably be housed within a protective, impact resistant and watertight enclosure 30a, such as plastic, for example, and can be selectively positioned within a pocket the main body of the backpack. Of course, any number of other analog and/or digital components capable of performing the described functionality can be provided in place of, or in conjunction with the described elements.

The processing unit 31 can include one or more central processing units (CPU) or any other type of device, or multiple devices, capable of manipulating or processing information such as program code stored in the memory 32 in order to allow the device to perform the functionality described herein.

Memory 32 can act to store operating instructions in the form of program code for the processing unit 31 to execute. Although illustrated in FIG. 3 as a single component, memory 32 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as a hard drive, for example. In the preferred embodiment, the memory can also function to temporarily or permanently store patient information inputted by the patient themselves (e.g., name, date of birth, medications, known health conditions) and patient data captured by the sensors.

The component interface unit 33 can function to provide a communicative link between the processing unit 31 and the sensors. In this regard, the component interface unit can include or can be connected to any number of different components such as one or more PIC microcontrollers, standard bus, internal bus, connection cables, and/or associated hardware capable of linking the various components.

The communication unit 34 can include any number of components capable of sending and/or receiving electronic signals with another device, either directly or over a network. The communication unit functions to allow a patient or practitioner to operate each of the sensors to collect patient data during a telemedicine session or other occurrence.

In one embodiment, communication unit can include or can be connected to one or more cable plugs 34a such as a USB port for example which can function to send and receive information with an external device such as a user smartphone 5 running a mobile application 15. The plug(s) can also function to receive power from an external power source in order to recharge the onboard battery or as the primary means of powering the device.

In one embodiment, the communication unit 34 can include a wireless transceiver for communicating wirelessly with an external device such as the above-described smartphone 5 running the mobile application 15, for example. To this end, any number of different types of transceivers such as a Wi-Fi, Bluetooth or cellular may be provided.

The power unit 35 can function to supply the required power to each of the system components. In one embodiment, the power unit can comprise a plurality of onboard batteries which are grouped together to form a rechargeable battery pack. The power unit can be linked with the above-described plug 34a, so as to also receive power from an external source such as the user's smartphone or other device.

The user interface 36 can function to receive and/or send operating instructions with a user directly via the controller device. To this end, the user interface can include any number of buttons 36a, and/or display screens 36b such as the illustrated touch screen, for example. Several nonlimiting examples of functions the user interface can perform include but are not limited to: turning the device ON or OFF, pairing the device with a wireless device, selectively operating one or more of the sensors, initiating a send or receive process with a linked device, and more, for example.

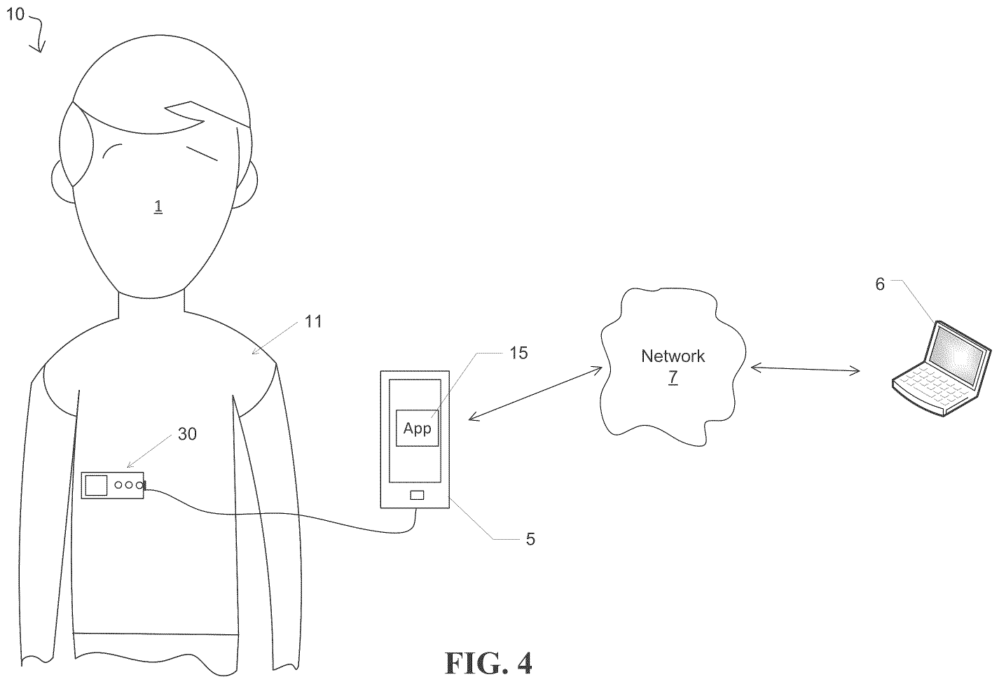

FIG. 4 illustrates one embodiment of the device 10 in operation. As shown, a patient 1 can position the garment over their chest so as to align and position each of the sensors in the manner described above. Next, the patient can physically or wirelessly connect the controller 30 to a processor enabled device such as a computer, tablet or smartphone 5, for example.

In one embodiment, the device 10 can include or communicate with a diagnostic garment application 15 which can be a mobile application that is downloaded and installed onto a user's processor enabled device 5. The application 15 can function to communicate with the controller 30 in order to allow the patient or other individual (such as a physician during an in-person examination) to selectively activate each of the sensors in order to receive patient data.

The information captured can be stored in the controller itself and/or uploaded to the device 5 for storage and/or transmission to other devices. In either instance, the sensor data will preferably be relayed in real time so as to allow the operator of the device to see and hear the patient information (e.g., sonogram images, sounds by the listening sensors, temperature readings, blood oxygen levels and more) as it is captured.

In one embodiment, the mobile application 15 can also include functionality for communicating with a healthcare provider interface device 6—e.g., computer, tablet, smartphone, etc.—over a network 7 such as the internet, for example. In this regard, the mobile application can include functionality for establishing or joining a voice and/or video communication session with the healthcare provider interface so as to allow the provider and patient to conduct a telemedicine visit. During such a visit, the application 15 and controller 30 will allow the healthcare provider interface device 6 to control each of the sensors, and to receive, view, listen and store the patient information in real time.

Although described above with regard to a mobile application for execution on a smartphone device, this is for illustrative purposes only, as the application can include virtually any type of instruction sets, in any form of programming language that can be executed on any type of processor enabled device (e.g., computer, tablet, smartwatch, etc.).

Moreover, some embodiments are contemplated wherein the controller 30 itself includes functionality for utilizing the onboard communication unit 34 to communicate directly or over a network with a healthcare provider interface device 6, to send patient information. Likewise, the controller may also include functionality for conducting a voice and/or video telemedicine visit with the device 6, without the aid of a separate user device 5. In such an embodiment, the controller would also include a speaker and microphone for use in conjunction with the display screen 36a, and actuation buttons 36b.

As to a further description of the manner and use of the present invention, the same should be apparent from the above description. Accordingly, no further discussion relating to the manner of usage and operation will be provided.

As described herein, one or more elements of the device 10 can be secured together utilizing any number of known attachment means such as sewing or gluing the sensors onto the fabric of the garment. Moreover, although the above embodiments have been described as including separate individual elements, the inventive concepts disclosed herein are not so limiting. To this end, one of skill in the art will recognize that one or more individually identified elements such as particular sensors may be formed together as a single sensor capable of performing multiple functions.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A medical diagnostic garment, said garment comprising:
a main body having a front panel, a back panel, and a neck area;
a plurality of sensors that are provided along an inside facing portion of the front panel; and
a controller that is communicatively linked to each of the plurality of sensors,
wherein each of the plurality of sensors are configured to capture patient data pertaining to a user wearing the main body.

2. The garment of claim 1, wherein the main body comprises:
a shirt that is configured to be worn along a torso of the user.

3. The garment of claim 1, wherein the plurality of sensors includes at least one electrode.

4. The garment of claim 3, wherein the patient data includes information about a heart of the user collected by the at least one electrode.

5. The garment of claim 1, wherein the plurality of sensors includes at least one pulse oxygen sensor.

6. The garment of claim 5, wherein the patient data includes pulse oxygen information of the user that is collected by the at least one pulse oxygen sensor.

7. The garment of claim 1, wherein the plurality of sensors includes at least one temperature sensor.

8. The garment of claim 7, wherein the patient data includes a body temperature of the user that is collected by at least one temperature sensor.

9. The garment of claim 1, wherein the plurality of sensors includes at least one ultrasound sensor.

10. The garment of claim 9, wherein the patient data includes a visual representation of an internal organ of the user that is collected by at least one ultrasound sensor.

11. The garment of claim 1, wherein the plurality of sensors includes at least one listening sensor.

12. The garment of claim 11, wherein the patient data includes sound produced by an internal organ of the user that is collected by at least one listening sensor.
